(19) United States
(12) Patent Application Publication
(10) Pub. No.: US 2002/0022022 A1
(43) Pub. Date: Feb. 21, 2002

(54) Inhibition of cell proliferation and matrix synthesis by antioxidants and NAD(P)H oxidase inhibitors

(76) Inventors: Yi Shi, Cheltenham, PA (US); Andrew Zalewski, Elkins Park, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 09/861,957

(22) Filed: May 21, 2001

Related U.S. Application Data
(60) Provisional application No. 60/206,001, filed on May 19, 2000.

(51) Int. Cl. A61K 38/44; A61K 31/198; A61K 38/06; A61K 31/40

(52) U.S. Cl. 424/94.4; 514/18; 514/423; 514/562

(57) ABSTRACT

The present invention is directed to a method for the prophylactic and therapeutic treatment of diseases or disorders associated with the abnormal proliferation and extracellular matrix synthesis of smooth muscle cells (SMC) and fibroblasts due to activation of NAD(P)H and/or increased ROS generation. The method involves the administration of an NAD(P)H oxidase inhibitor(s) and/or antioxidant(s) to a mammal in an amount sufficient to treat the disease or disorder prophylactically or therapeutically. The NAD(P)H oxidase inhibitor inhibits the synthesis or translocation of NAD(P)H subunits, thereby blocking the generation of intracellular reactive oxygen species (ROS) and thus the proliferation and extracellular matrix synthesis of SMC and fibroblasts. Similarly, the administration of antioxidants blocks the generation of intracellular ROS, thereby inhibiting SMC and fibroblast proliferation and extracellular matrix synthesis. In addition to the prevention and treatment of vascular disease, such as atherosclerosis, graft disease, and restenosis, NAD(P)H oxidase inhibitors and antioxidants may be useful for the prevention and treatment of other conditions by decreasing cell proliferation and extracellular matrix synthesis associated therewith. These conditions include arthritis, keloid formation, cancer, tissue and organ fibrosis, and complications related to organ transplantation, metabolic syndrome, and radiation therapy.

### CONTINUING APPLICATION DATA

This application claims priority under 35 U.S.C. § 119 based upon U.S. Provisional Application No. 60/206,001 filed May 19, 2000.

### GOVERNMENT RIGHTS TO THE INVENTION

The invention was made with government support under grants HL-44150 and HL-60672 awarded by the National Institutes of Health. The government has certain rights to the invention.

### FIELD OF THE INVENTION

The present invention relates to the fields of molecular biology and cardiology, and to a method of treating diseases or disorders associated with the abnormal proliferation of cells and extracellular matrix synthesis associated with the activation of NAD(P)H oxidase and the increased generation of intracellular reactive oxygen species (ROS) and, more particularly, to the blocking of the generation of intracellular ROS.

### BACKGROUND OF THE INVENTION

Oxidative stress is an important modulator of vascular cell function and has been implicated in several steps leading to the development of vascular disease. (1,2) Initial observations focused on reactive oxygen species (ROS) derived from invading macrophages and their possible involvement in oxidative lipid modifications in the vessel wall. Subsequently, it has become apparent that ROS also are produced in a controlled fashion by all vascular cells and that they act as “second messengers,” regulating various cellular functions. Several extracellular signals, such as growth factors or even physical stimuli, induce ROS and their derivatives in vascular smooth muscle cells (SMC) and fibroblasts, activating the intracellular growth program. (3,4,5,6) For example, superoxide anion (—O₂⁻) increases expression of ERK½ MAP kinase, whereas H₂O₂ activates p38 MAP kinase and stress-activated proteins. (7,8) Furthermore, H₂O₂ also stimulates early proto-oncogenes and redox-sensitive transcriptional factors (e.g., NF-kB and AP-1). (9)

In vascular cells, the major enzymatic source of intracellular ROS is NAD(P)H oxidase, which generates —O₂⁻ by one-electron reduction of molecular oxygen. (10,11,12) Although NAD(P)H oxidase is responsible for the burst of —O₂⁻ in phagocytic cells, the generation of ROS in vascular cells differs from that in neutrophils. In the former, it occurs over a period of hours (rather than minutes), appears to be mostly intracellular (rather than extra- and intracellular) and may involve the assembly of different enzymatic subunits of NAD(P)H oxidase. (13) Significant progress has been made toward the identification of NAD(P)H oxidase subunits in normal vascular cells and in atherosclerotic lesions, including both membrane-associated (p22^{phox}) and cytoplasmic components (p67^{phox}, p47^{phox}, Rac1). (6,14,15,16) The activity of NAD(P)H oxidase in vascular cells is modulated by extracellular signals known to influence vascular remodeling and lesion development (e.g., thrombin and angiotensin II). (6,14,15,16,17) Furthermore, gene polymorphism affecting at least one of the subunits (p22^{phox}) has been linked to the development of atherosclerosis in humans. (18,19)

The regulation of the redox state appears to be heterogeneous across the vessel wall. Higher expression of NAD(P)H oxidase and —O₂⁻ production have been reported in normal adventitia, as compared with the media. (20,21) The importance of this finding initially remained unclear, since the activation of medial SMC and lipid peroxidation occur in the proximity of the arterial lumen. Several studies, however, have suggested active involvement of adventitial fibroblasts in arterial repair. (22,23,24) In particular, after severe coronary injury, these cells demonstrate preferential proliferation and migration toward intima. This is not surprising since coronary SMC display more advanced differentiation and a limited response to stimulation as compared with non-coronary SMC. (25,26) In view of these findings and the established role of ROS in the regulation of cell proliferation, it is hypothesized that the increase in oxidative stress after coronary injury involves adventitial fibroblasts.

The results of the present invention demonstrate the upregulation of NAD(P)H oxidase activity and ROS production in adventitial fibroblasts after coronary injury. In cell culture, ROS are important signals for growth response of coronary fibroblasts. The evidence of the present invention shows that phenotypic responsiveness of coronary fibroblasts to stimulation is mediated, in part, by NAD(P)H oxidase derived oxidative stress.

It is well recognized that saphenous vein grafts (SVG) demonstrate lower patency rates compared with arterial grafts (AG) in patients that undergo surgical coronary revascularization. (43) Early attrition of SVG has been attributed to thrombosis and rapid neointimal formation within the first year after surgery. After a period of clinical quiescence, the loss of SVG patency resumes due to graft atherosclerosis manifested by occlusive lesions beginning at 3-5 years after revascularization. Neither better preoperative patient selection nor improved intraoperative handling of vascular conduits have been sufficient to eliminate the disparity between SVG and AG. (44,45) Recent studies increasingly have focused on biological differences between venous and arterial conduits that may affect their response to surgery. (46,47) Besides structural differences (e.g., less developed elastic tissues), cellular composition of veins is dissimilar when compared with arteries. “Non-muscle” fibroblasts, which are typically present in the adventitia of normal vessels, are common in the media of saphenous veins. (48,49) These poorly differentiated cells are highly proliferative, and their population is further augmented by migration of adventitial and perivascular fibroblasts through the injured media. (49) In contrast, SMC of the arterial conduit exhibit less cellular activation, while adventitial fibroblasts are prevented from transmural migration by intact elastic tissues in AG.

Under normal conditions, —O₂⁻ is rapidly inactivated by superoxide dismutase (SOD) stored in the extracellular matrix of the tunica media. (50,51) Interestingly, fibroblast-rich adventitia generates more —O₂⁻ than medial SMC, although the importance of this phenomenon has not been fully explained. (20,21) The differences in cellular composition between SVG and AG raise the possibility that the activation of fibroblasts and/or the loss of SOD activity result in the increase in oxidative stress in the media of SVG. The results of the present invention demonstrate that SVG and AG exhibit dissimilar oxidative stress, lipoprotein accumulation, and oxidative modification of retained low density lipoproteins (LDL). These findings illustrate that early changes during SVG remodeling contribute to SVG attrition due to accelerated atherogenesis.

The present invention provides a method for inhibition of cell proliferation and extracellular matrix synthesis that is due to the activation of NAD(P)H oxidase and increased generation of intracellular ROS.

### ABBREVIATIONS

“ROS” means “reactive oxygen species”

“SMC” means “smooth muscle cells”

“SVG” means “saphenous vein grafts”

“AG” means “arterial grafts”

“SOD” means “superoxide dismutase”

“LDL” means “low density lipoproteins”

“NBT” means “nitroblue tetrazolium”

“SM-MHC” means “smooth muscle myosin heavy chain”

“DMEM” means “Dulbecco's modified Eagle's medium”

“iNOS” means “inducible nitric oxide synthase”

“DDT” means “dry, defatted tissue”

“WW” means “wet weight”

“DPI” means “diphenyleneiodonium”

“GAG” means “glycosaminoglycan”

### DEFINITIONS

“Prophylactic” as used herein means the protection, in whole or in part, against diseases, disorders, and conditions associated with the abnormal proliferation of cells and extracellular matrix synthesis associated with the activation of NAD(P)H oxidase and generation of ROS.

“Therapeutic” as used herein means the amelioration of, and the protection, in whole or in part, against further, diseases, disorders, and conditions associated with the abnormal proliferation of cells and extracellular matrix synthesis associated with the activation of NAD(P)H oxidase.

“NAD(P)H subunits” as used herein include, but are not limited to, p22^{phox}, gp91^{phox}, Nox-1, p47^{phox}, and p67^{phox}.

### BRIEF DESCRIPTION OF THE FIGURES

FIG. 1. Superoxide anion (—O₂⁻) generation in coronary adventitia and media in control (uninjured) arteries. The adventitia show higher basal level of —O₂⁻ production compared with the media (SOD-inhibitable NBT reduction). A NAD(P)H oxidase inhibitor, diphenyleneiodonium (DPI), inhibits —O₂⁻ generation in coronary adventitia, whereas oxypurinol (OXY) and rotenone (ROT) show no inhibitory effects. The lack of SOD-inhibitable NBT reduction in boiled adventitia further confirms enzymatic source of —O₂⁻ production. *: p<0.001 vs. coronary media; t: p<0.001 vs. coronary adventitia without treatment (n=6-9 vascular rings/bar).

FIG. 2. The time course of —O₂⁻ production after coronary injury. The —O₂⁻ production (SOD- and tiron-inhibitable NBT reduction) is measured in transmural segments of uninjured and injured vessels. The increase in —O₂⁻ generation is observed within 1 day after injury. *: p<0.05 and t: p<0.01 vs. uninjured coronary arteries. Numbers represent the number of vascular rings.

FIG. 3. Localization of oxidative stress in injured coronary arteries. Injured coronary arteries (2 days) are incubated with NBT for 3 hours and processed for NBT histology. A: A cross section of injured coronary artery with apparent site of injury. The areas of the adventitia and media outlined by rectangular boxes are depicted at higher magnification. B: Intracellular deposits of formazan (blue) are present in adventitial cells in the center of injury. C: Coronary medial SMC show no intracellular formazan deposits despite medial dissection. Magnifications: A: ×40; B and C: ×410; Abbreviation: a: adventitia; m: media; NBT: nitroblue tetrazolium.

FIG. 4. Localization of NAD(P)H oxidase subunits in injured coronary arteries. A: A cross section of injured coronary artery stained for SM-MHC. At 2 days after injury, coronary medial SMC exhibit a strong SM-MHC immunoreactivity, whereas adventitial cells are negative. The areas of the adventitia and media outlined by rectangular boxes are depicted at higher magnification. B: Injured adventitia contain mostly replicating fibroblasts with only infrequent macrophages. C: The majority of activated adventitial cells exhibit increased immunoreactivity for p47^{phox}. D: Coronary media show no increase in p47^{phox} immunoreactivity. E: Likewise, p67^{phox} is increased in adventitial cells, but not in the media (not shown). F: Negative control (primary antibody is omitted). SM-MHC: smooth muscle myosin heavy chain; m: media; a: adventitia; NC: negative control. Magnification: A ×40, B-E: ×41 0.

FIG. 5. Serum-induced —O₂⁻ generation in coronary fibroblasts. Adventitial fibroblasts are plated in 6-well plate at 100,000 cells/well in 10% FBS for 2 days. The cells are growth arrested with 0.5% FBS for 48 hours followed by stimulation with 10% FBS. The NBT is added to cells for one hour and intracellular accumulation of formazan is measured. Adventitial fibroblasts demonstrate a time dependent —O₂⁻ production peaking at 6 hours after stimulation (n=9/time point). *: p<0.001 vs. no stimulation.

FIG. 6. Inhibition of serum-induced —O₂⁻ generation in coronary fibroblasts. Adventitial fibroblasts are pretreated with DPI (10 μM) or SOD (500 U/ml) for 30 minutes followed by stimulation with 10% FBS for 3 hours. The cells then are incubated with NBT for one hour and the inhibition of formazan accumulation is measured. Both DPI and SOD show significant inhibition on —O₂⁻ production (n=4/treatment). *: p<0.001 vs. 10% FBS.

FIG. 7A. ROS and adventitial fibroblast proliferation. Adventitial fibroblasts (10,000 cells/well) are arrested with 0.5% FBS for 48 hours and then stimulated with 10% FBS in the presence of various inhibitors. Cell growth is examined at 3 days after treatment by cell counting. The inhibitor of NAD(P)H oxidase (DPI), the scavenger of —O₂⁻ (tiron), or the removal of H₂O₂ (CAT) significantly inhibits fibroblast growth in a concentration-dependent manner. In contrast, dismutation of —O₂⁻ to H₂O₂ with SOD shows no significant inhibition of fibroblast proliferation. *:p<0.05 and †: p<0.01 vs. 10% FBS alone (n=3/bar). The experiments are repeated 3 times yielding similar results.

FIG. 7B. ROS and vascular SMC proliferation. SMC (10,000 cells/well) are arrested with 0.5% FBS for 48 hours and then stimulated with 10% FBS in the presence or absence of various inhibitors. Cell growth is examined at 3 days after treatment by cell counting. The inhibitors of NAD(P)H oxidase (DPI and apocynin), the scavengers of —O₂⁻ (NAC, PDC, and tiron), or the removal of H₂O₂ (CAT) significantly inhibits SMC growth in a concentration-dependent manner. In contrast, dismutation of —O₂⁻ to H₂O₂ with SOD shows no significant inhibition of SMC proliferation. *:p<0.05 and †: p<0.01 vs. 10% FBS alone (n=3/bar). The experiments are repeated 3 times yielding similar results.

FIG. 8. Superoxide anion production in normal vessels and in vascular grafts. Superoxide (SOD-inhibitable NBT reduction) is measured at 2 weeks after surgery as described infra. Saphenous veins and SVG generate significantly more superoxide than normal arteries and AG, respectively. *denotes p<0.01 vs. normal carotid artery; † denotes p<0.01 vs. AG; n=5-7/bar.

FIG. 9. Superoxide anion is significantly reduced in the presence of NAD(P)H oxidase inhibitor, diphenyleneiodonium (DPI, 100 μM). The inhibitors of xanthine oxidase, mitochondrial dehydrogenase, and nitric oxide synthase do not decrease SOD-inhibitable NBT reduction. * denotes p<0.01 vs. all other groups; OXY: oxypurinol (300 μμM); ROT: rotenone (50 μμM); L-NAME: N^{ω}-nitro-L-arginine methyl ester (1 mM); n=3-6/bar.

FIG. 10. SOD activity in normal vessels and in vascular grafts. SOD (SOD-dependent inhibition of cytochrome c reduction catalyzed by xanthine/xanthine oxidase) is assessed at 2 weeks after surgery as described infra. SVG demonstrate significant reduction in SOD activity compared to AG. *denotes p<0.001 vs. AG; n=5-8/bar.

FIG. 11. Versican immunoreactivity in SVG. A: Normal saphenous vein shows a small area of positive staining in the subendothelial region with the media lacking versican; B: SVG exhibit marked increase in extracellular versican, which is present in the intima and media (not shown) at 2 weeks after surgery. Magnification ×410.

FIG. 12. Lipid retention in vascular grafts from hyperlipemic animals at 1 month after surgery. A: AG show preserved elastic tissues and no significant intima (Verhoeffs stain). The rectangular box identifies area shown in B (serial sections); B: No significant lipid accumulation is noted in AG (Red-O-stain); C: SVG with prominent neointima (n) and remodeled media (m) (Verhoeffs stain). The rectangular box identifies area shown in D-H (serial sections); D: Focal lipid retention in the neointima (Red-O-stain); E-G: Lipid positive regions contain apoB, oxidized epitopes, and versican; H: negative control (N/C) stained without primary antibody. Magnifications: A and C: ×20 and B, D-H: ×200.

### DETAILED DESCRIPTION OF THE INVENTION

The present invention provides a method for inhibition of cell proliferation and extracellular matrix synthesis associated with NAD(P)H activation and ROS generation. The method comprises administering a therapeutically effective amount of an NAD(P)H oxidase inhibitor, including, but not limited to, diphenyleneiodonium (DPI) and 4-hydroxy-3-methoxyacetopoenone (apocynin) or any substance that inhibits synthesis or translocation of NAD(P)H oxidase subunits (p22^{phox}, gp91^{phox}, Nox-1, p47^{phox}, and p67^{phox}), or an antioxidant, such as, but not limited to, N-acetylcysteine (NAC), pyrrolidinedithiocarbamate (PDTC), tiron, catalase, and glutathione, in order to block the generation of intracellular ROS in fibroblasts and SMC. The invention, therefore, is useful in the treatment of vascular disease, including, but not limited to, atherosclerosis, restenosis after revascularization procedures, and graft disease, as well as nonvascular diseases that are caused by abnormal proliferation and matrix synthesis of fibroblasts and SMC. The invention further can be applied to any disease or disorder involving abnormal growth of cells and matrix synthesis of cells associated with the activation of NAD(P)H oxidase, including, but not limited to, keloids, tissue and organ fibrosis, inflammatory disease (e.g., arthritis), and complications related to cancer, organ transplantation, metabolic syndrome, and radiation therapy,

While this invention is described with a reference to specific embodiments, it will be obvious to those of ordinary skill in the art that variations in these methods and compositions may be used and that it is intended that the invention may be practiced otherwise than as specifically described herein. Accordingly, this invention includes all modifications encompassed within the spirit and scope of the invention as defined by the claims.

### EXAMPLE 1

### Increased NAD(P)H oxidase and ROS in coronary arteries after balloon injury

Methods

### Animal model

Domestic crossbred female pigs (12-15 kg) are anesthetized and instrumented, as previously described. (22,23) After the exposure of the right common carotid artery, heparin (5,000 U) is intravenously administered. Coronary ostia are cannulated under fluoroscopic guidance and intracoronary nitroglycerin is given (100 μg). Coronary injury is carried out in two coronary arteries in each animal using an oversized balloon (balloon:artery ratio ˜1.3-1.5) inflated 2-3 times for 30 seconds. The third artery is used as a control. The animals are euthanatized with intravenous Euthasol (Delmarva Laboratory) at the times indicated in the text.

### Measurement of —O₂⁻ production

The production of —O₂⁻ is measured by superoxide dismutase- (SOD) or tiron-inhibitable conversion of nitroblue tetrazolium (NBT) to formazan. (20,21)

—O₂⁻ production in coronary arteries: Coronary arteries are dissected free from adipose tissue and myocardium and then cut into ˜5 mm rings and placed in 24-well plates. Tissues are balanced in phenol-free DMEM at 37° C. in a CO₂ incubator for 30 minutes with or without addition of SOD (SOD, 1000 unit/ml) or tiron (10 mM), both scavengers of —O₂⁻ Freshly made NBT (100 mg/L in phenol-free DMEM) is added to the tissues with a gentle rocking for 3 hours. The reaction is terminated by addition of equal volume of 0.5N HCl, and tissues are rinsed twice with cold PBS. To extract formazan, tissues are pulverized in liquid nitrogen and dissolved in 100% pyridine at 80° C. for 30 minutes. After centrifugation, light absorbance is read in supernatants at 540 nm. The NBT reduction to formazan is calculated using the following formula: NBT reduction=A×V/(T×E×L) (A: absorbance; V: volume of solubilizing solution; T: time of incubation with NBT (minutes); E: extinction coefficient=0.72 mmol.mm; L: length of light travel through the solution, 10 mm). Either SOD- or tiron-inhibitable NBT reduction is calculated as measures of —O₂⁻ production (pmol/min/mg wet weight). To determine the pathways mediating —O₂⁻ production, several inhibitors are used in the experiments, including diphenyleneiodonium (DPI, 100 μM), rotenone (ROT, 50 μM), oxypurinol (OXY, 300 μM), N^{ω}-nitro-L-arginine methyl ester (L-NAME, 1 mM), and diethyldithio-carbamic acid (DETCA, 10 mM). The n value represents the number of vascular rings obtained from at least 3 animals per experimental condition.

To assess the location of injury-induced —O₂⁻ production, the injured coronary arteries are incubated with NBT and processed to visualize formazan deposits. Briefly, coronary rings are fixed in 10% formalin and embedded in paraffin. They are sectioned into 6-μm-thick sections and deparaffinized by heating at 65° C. for one hour. To avoid solubilization of NBT in tissue, the sections are rinsed with Clear-Rite 3 solution (Richard-Allan Scientific) and counterstained with Nuclear Fast Red.

—O₂⁻ production in isolated adventitial fibroblasts: Adventitial fibroblasts (passage 2-6) are plated in 6-well plates at 100,000 cells/well in 10% FBS. Two (2) days later, when cells are ˜80% confluent, they are arrested in 0.5% FBS for the next 48 hours. Afterwards, they are stimulated with 10% FBS for 1-24 hours, followed by incubation with NBT (0.5 mg/ml in phenol-free DMEM) for one hour. After a brief washing, cells are trypsinized, and cell pellets are dissolved in 100% pyridine. The light absorbance is measured at 540 nm, and the NBT reduction to formazan is calculated as described above and corrected by cell number. Values are derived from 6-9 wells from three separate experiments.

### Measurement of NAD(P)H oxidase activity in coronary arteries

NAD(P)H oxidase activity is measured by SOD inhibitable cytochrome c reduction using NADH or NAD(P)H as substrates. (17) To measure NAD(P)H oxidase activity in injured coronary arteries, the arteries are harvested at 2 days after injury, and the injured segments (including the adventitia and media) are dissected free from adipose tissue and myocardium. The non-instrumented coronary arteries are used as control. After the removal of endothelial cells, tissues are minced in 10-volume of ice-cold Tris-sucrose buffer (pH 7.1) containing Tris base (10 mM), sucrose (340 mM), PMSF (1 mM), EDTA (1 mM), leupeptin (10 μg/ml), aprotinin (10 μg/ml), and pepstatin (10 μg/ml). Then the tissue homogenates are sonicated for 20 seconds on ice, followed by extraction for 30 minutes. After centrifugation at 15,000 g for 10 minutes, an aliquot (20 μl) of supernatant (50-150 μg of protein) is added to a reaction buffer (980 μl) containing cytochrome c (78 μM) and NADH or NAD(P)H (100 μM), with or without SOD (1000 U/ml). The samples then are incubated at 37° C. for one hour, and the absorbance at 550 nm is measured. There is no measurable activity in absence of NADH. A buffer blank is measured in each assay and SOD inhibitable cytochrome c reduction in the buffer blank is subtracted from each sample. The activity of NAD(P)H or NADH oxidase is calculated as SOD inhibitable cytochrome c reduction and expressed as —O₂⁻ pmol /mg/min.

### Measurement of SOD activitv in coronary arteries

SOD activity in vascular tissues is measured by SOD-dependent inhibition of cytochrome c reduction catalyzed by xanthine/xanthine oxidase. (27) To assess SOD activity in uninjured coronary arteries, coronary adventitia and media are dissected after the removal of endothelium. SOD activity after coronary injury is measured in arterial segments including the adventitia and media. The tissues are minced and homogenized in 10-volume of 50 mM potassium phosphate (pH 7.4) containing 0.3 M KBr and a cocktail of protease inhibitors (0.5 mM PMSF, 90 μg/ml aprotinin, 10 μg/ml pepstatin, 10 μg/ml leupeptin). After sonication for 20 seconds, the homogenates are extracted at 4° C. for 30 minutes followed by centrifugation at 15,000 g for 10 minutes. The supernatants are added to a reaction mixture consisting of 0.1 mM EDTA, 0.090 mM xanthine, and 0.018 mM cytochrome c (pH 7.4). SOD activity is assessed by monitoring the inhibition of xanthine oxidase-mediated cytochrome c reduction with the absorbance measured at 550 nm over 3 minutes.

### Immunohistochemistry

The Vectastain Elite ABC system (Vector Laboratories) is used for immunohistochemistry as previously described. (22) Sections are deparaffinized, incubated with 0.6% hydrogen peroxide in methanol for 30 minutes, and blocked with 5% horse or rabbit serum. After washing in PBS, sections are incubated with primary antibodies for 1 hour at room temperature in a moisture chamber. The following primary antibodies are used: polyclonal antibodies against p47^{phox} and p67^{phox} (1:200, Santa Cruz), monoclonal antibody recognizing smooth muscle myosin heavy chain (SM-MHC, 1:800, Sigma), and porcine macrophages (1:10, ATCC HB 142). Afterwards, slides are washed and incubated with biotinylated secondary horse anti-mouse or rabbit anti-goat antibodies (1:2000, Vector Laboratories) for 1 hour. They are visualized with DAB substrate (Vector Laboratories) followed by counterstain with Gill's hematoxylin (Sigma Diagnostics). Negative controls are carried out using nonimmune serum instead of primary antibody.

### Cell proliferation assay

Fibroblasts are isolated from the adventitia of porcine coronary arteries as described. (26) The cells (passage 2-6) are plated in triplicates at 10,000 cells/well in 24-well plates in DMEM supplemented with 10% FBS. At 24 hours later, cells are arrested in DMEM containing 0.5% FBS for 48 hours. Afterwards, they are stimulated with 10% FBS for 3 days with or without addition of indicated inhibitors. Cells are trypsinized at 72 hours after stimulation and counted in a Coulter counter. Values are derived from 3 wells/treatment and the experiments are repeated at least three times on separate occasions.

### Statistical analyses

Data are expressed as mean±SD. The statistical significance regarding multigroup comparisons is determined using ANOVA with Bonferroni correction. A value of p<0.05 is considered significant.

### Results

### Oxidative stress in normal and injured coronary arteries

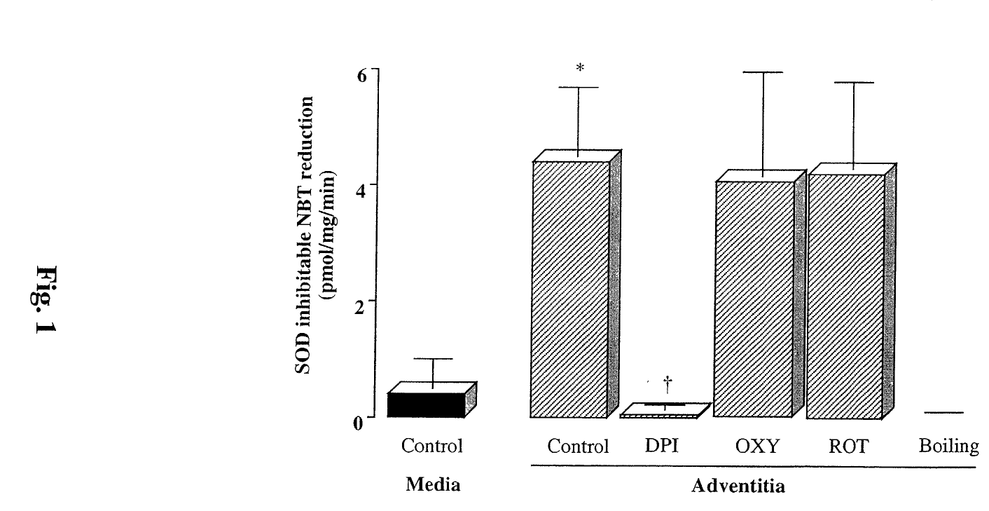

Normal coronary arteries. Normal coronary adventitia exhibit higher basal —O₂⁻ generation (4.4±1.2 pmol/mg/min SOD-inhibitable NBT reduction), as compared with the media (0.4±0.5 pmol/mg/min, n=6, p<0.01). As shown in FIG. 1, the pre-incubation of coronary rings with DPI (NAD(P)H oxidase inhibitor), abolishes —O₂⁻ production in coronary adventitia, whereas ROT (mitochondrial dehydrogenase inhibitor) and OXY (xanthine oxidase inhibitor) have no effect. The difference in basal —O₂⁻ generation between coronary adventitia and media is likely due to heterogeneous distribution of endogenous SOD, inasmuch as adventitia exhibit lower SOD activity compared with the media (101±7 vs. 166±9 U/g, p<0.001, n=5). Subsequently, SOD inhibitor (DETCA) augments more —O₂⁻ generation in the media (adventitia: 19.6±2.5 and media: 36.3±8.2 pmol/mg/min, p<0.01 vs. no DETCA).

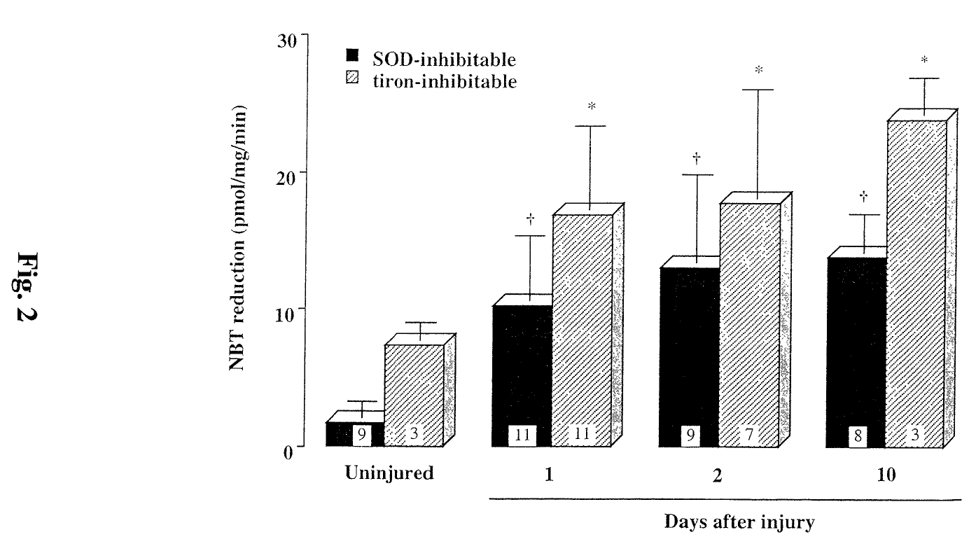

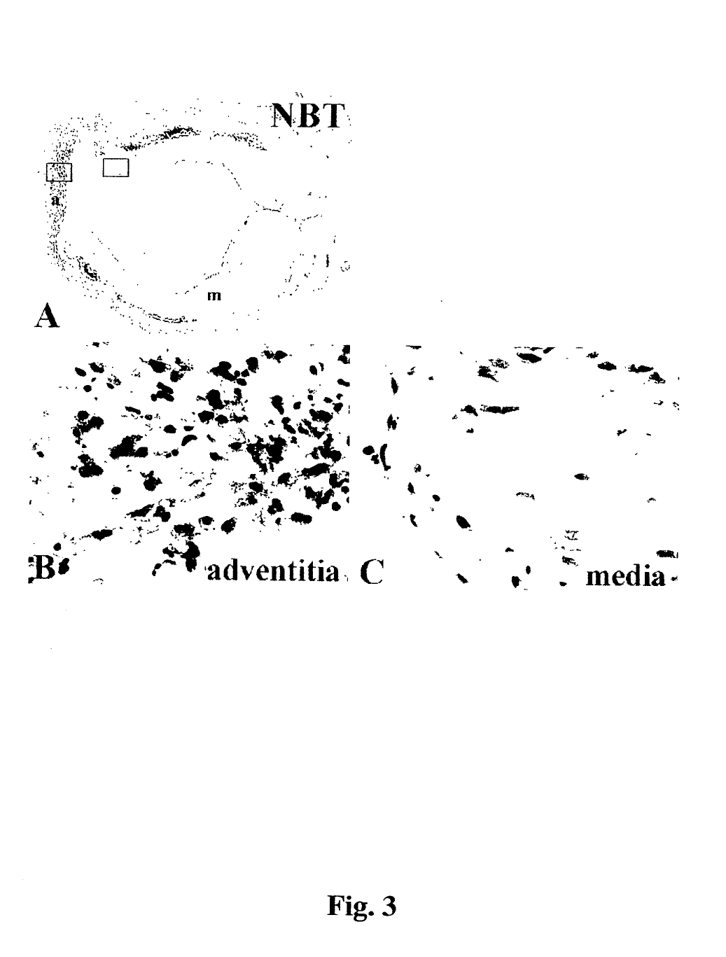

Injured coronary arteries. Since coronary injury induces a short-lived adventitial cell proliferation, the change in oxidative stress during this time period is examined. To this end, the SOD activity and —O₂⁻ generation are measured in the entire coronary segment since precise separation of the adventitia from media is not technically feasible at early time points after injury. SOD activity shows no difference between control and injured coronary segments. —O₂⁻ generation, as measured by SOD- and tiron-inhibitable NBT reduction, significantly increases within 1 day after injury and remains elevated for at least 10 days (FIG. 2). Higher values of tiron-inhibitable NBT reduction are due to better cellular permeability of tiron as compared with SOD. To ascertain the site of —O₂⁻ generation in injured vessels, reduced NBT (formazan) is identified in cross sections. FIG. 3 demonstrates preferential adventitial localization of intracellular deposits of formazan in injured segments. Similar to uninjured vessels, NAD(P)H oxidase inhibitor (DPI) almost entirely abolishes the production of —O₂⁻ after coronary injury (n=4/time point, p<0.001 vs. no treatment). Although dynamic changes in inducible nitric oxide synthase (iNOS) expression during coronary repair contribute to oxidative stress, its inhibitor, L-NAME, shows no effect (Table 1).

TABLE 1
NAD(P)H oxidase dependent superoxide
generation in injured coronary arteries.

| Time | Superoxide generation | | |
|---|---|---|---|
| After Injury | Control | DPI | L-NAME |
| 2 days | 14.8 ± 7 | 0.5 ± 0.9* | 12.9 ± 9.4 |
| 10 days | 13.8 ± 2.7 | 0.2 ± 0.3* | 11.5 ± 6.1 |

Superoxide anion is measured as SOD-inhibitable NBT reduction (pmol/mg/min). Vascular rings derived from injured coronary arteries are pretreated without or with DPI (100 μM) or L-NAME (1 mM) for 30 minutes. NAD(P)H oxidase inhibitor, DPI, significantly inhibits superoxide generation, whereas iNOS inhibitor, L-NAME, shows no effects. *: p < 0.001 vs. control, n = 4/timepoint.

### NAD(P)H oxidase activity and expression of subunits

NAD(P)H oxidase activity. To ascertain if NAD(P)H oxidase is the major pathway responsible for oxidative stress after coronary injury, NAD(P)H oxidase activity is measured by SOD inhibitable cytochrome c reduction using NADH or NAD(P)H as substrates. At baseline, coronary arteries exhibit similar levels of NADH and NAD(P)H oxidase activity. At 2 days after coronary injury, NADH oxidase activity is significantly augmented in the injured and adjacent injured segments (Table 2), whereas NAD(P)H oxidase activity shows no changes after coronary injury.

TABLE 2
NAD(P)H oxidase activity in injured coronary arteries.

| | NADH | NAD(P)H |
|---|---|---|
| Uninjured (n = 6) | 7.7 ± 5.4 | 10.3 ± 8.2 |
| Injured (n = 10) | 27.0 ± 4.4*† | 19.8 ± 7.1 |
| Adjacent (n = 5) | 17.1 ± 2.3* | 16.9 ± 5.9 |

NADH oxidase activity is measured in uninjured and injured coronary segments at 2 days after the injury. Injury significantly augments NADH oxidase activity (O₂ pmol/mg/min), whereas NAD(P)H oxidase activity shows no major changes as compared with uninjured coronary arteries. *: p < 0.01 vs. uninjured coronary arteries. †: p < 0.01 vs. adjacent coronary arteries. Numbers in parenthesis represent the number of vessels.

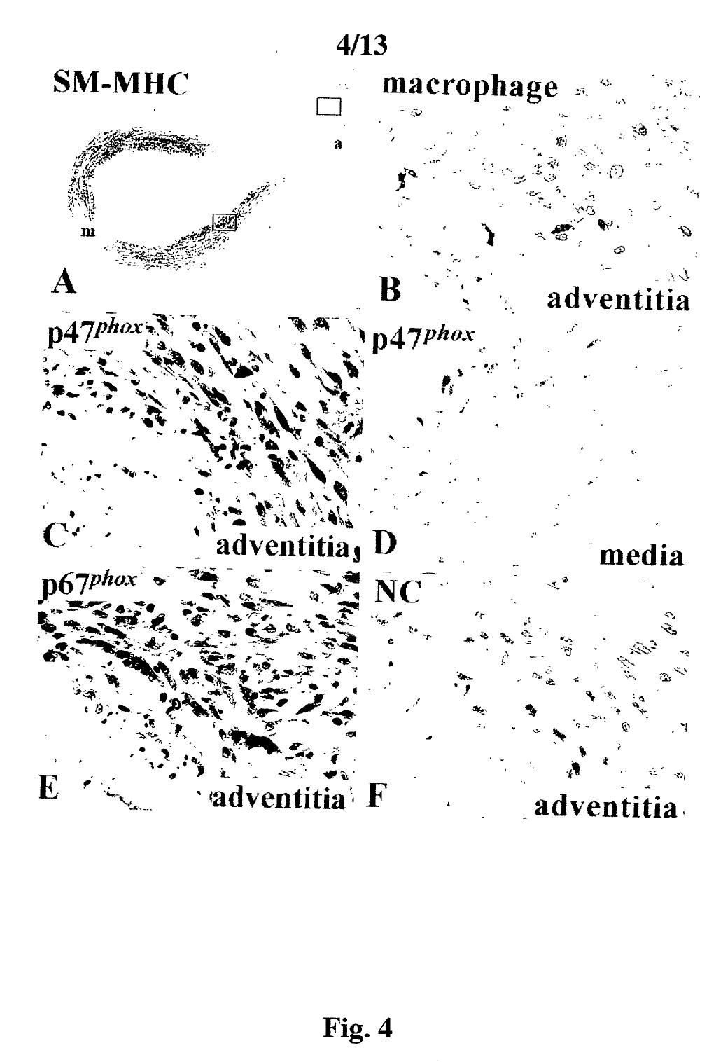

Expression of p47^{phox} and p67^{phox}. To localize NAD(P)H oxidase subunits in injured coronary arteries, expression of p47^{phox} and p67^{phox} (cytoplasmic subunits of NAD(P)H oxidase) is examined by immunohistochemistry. Expression of p47^{phox} and p67^{phox} is low in normal coronary arteries but shows a marked increase in adventitial cells after injury. The expression begins at day 1 and peaks 2 days after injury. Positive cells are of fibroblastic origin since they lack SMC differentiation markers (SM myosin heavy chain, α-SM actin, desmin, and caldesmon) and only infrequent cells (<5%) are positive for macrophage immunoreactivity (FIG. 4).

### Role of NAD(P)H oxidase-derived ROS production in vascular cell proliferation

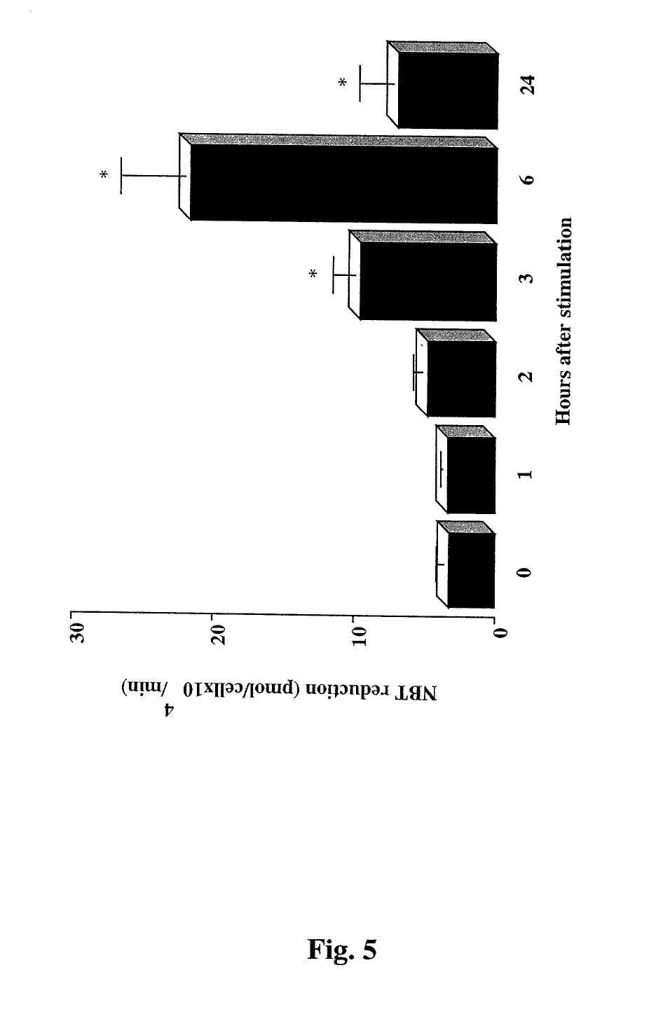

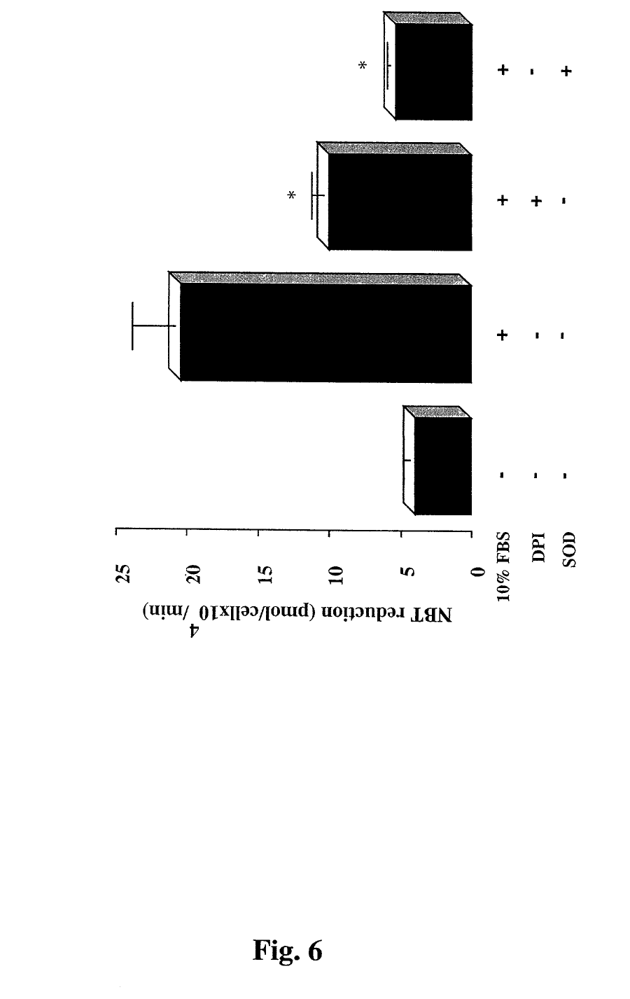

Serum-induced superoxide production in vascular fibroblasts. To assess the functional importance of increased oxidative stress in vascular cells, the —O₂⁻ production is examined in serum-stimulated fibroblasts and SMC. In response to serum stimulation, fibroblasts and SMC demonstrate time-dependent increase in —O₂⁻ production, reaching maximum levels at 3-6 hours (FIG. 5). As expected, either the inhibition of NAD(P)H oxidase with DPI (10 μM) or dismutation of —O₂⁻ with exogenous SOD (500 U/ml) produces significant reduction in —O₂⁻ production (FIG. 6), whereas L-NAME, ROT, and OXY show no effects.

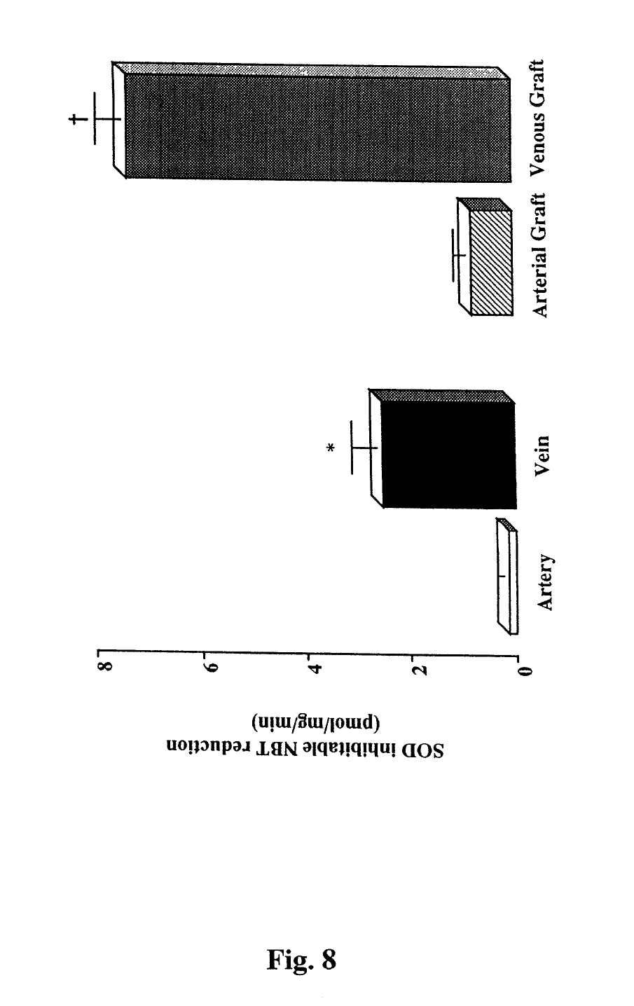

Serum-induced superoxide generation and vascular cell proliferation. To assess whether altering ROS generation could modulate vascular cell proliferation in vitro, growth inhibition of serum stimulated cells is determined either by inhibiting the generation of ROS (DPI) or facilitating their removal (—O₂⁻ tiron, SOD and H₂O₂: CAT). The inhibitor of NAD(P)H oxidase (DPI) significantly inhibits cell growth in a concentration-dependent manner (FIG. 8, p<0.001). In contrast, L-NAME and OXY produce no significant effects. These results are consistent with the lack of inhibition of ROS generation by these inhibitors. The removal of either —O₂⁻ with tiron or H₂O₂ with CAT inhibits cell proliferation. In contrast, dismutation of —O₂⁻ to H₂O₂ after SOD does not prevent serum-induced cell replication.

### Discussion

The present invention provides evidence that: (1) coronary adventitia is an important source of increased oxidative stress after endoluminal coronary injury; (2) NAD(P)H oxidase is the major pathway for ROS generation in injured coronary arteries and stimulated adventitial fibroblasts; and (3) ROS are involved in the regulation of growth response of both vascular fibroblasts and SMC.

Oxidative stress is known to increase after various forms of vascular insult. (6,29,30) Although the presence of NAD(P)H oxidase has been shown in normal adventitia (16,20,21), its role in cellular proliferation during arterial repair previously has not been elucidated. In non-coronary vasculature, there is a rapid decrease in glutathione level, an indirect marker of the redox state, after mechanical injury. (30) Others have reported the induction of p47^{phox}, thus implicating NAD(P)H oxidase and ROS generation in initial SMC proliferation. (6) Likewise, p22^{phox} expression and oxidative stress are increased in aortic medial SMC after angiotensin II infusion. (31) Unique characteristics of coronary SMC, however, raise the question whether similar events occur during coronary repair. (25,26) Earlier studies showed an increase in —O₂⁻ production at 2 weeks after coronary injury; the source of the increase, however, could not be identified due to the presence of the neointima, which contains cells of adventitial and medial origin, blood-borne cells, and regenerating endothelial cells. (29)

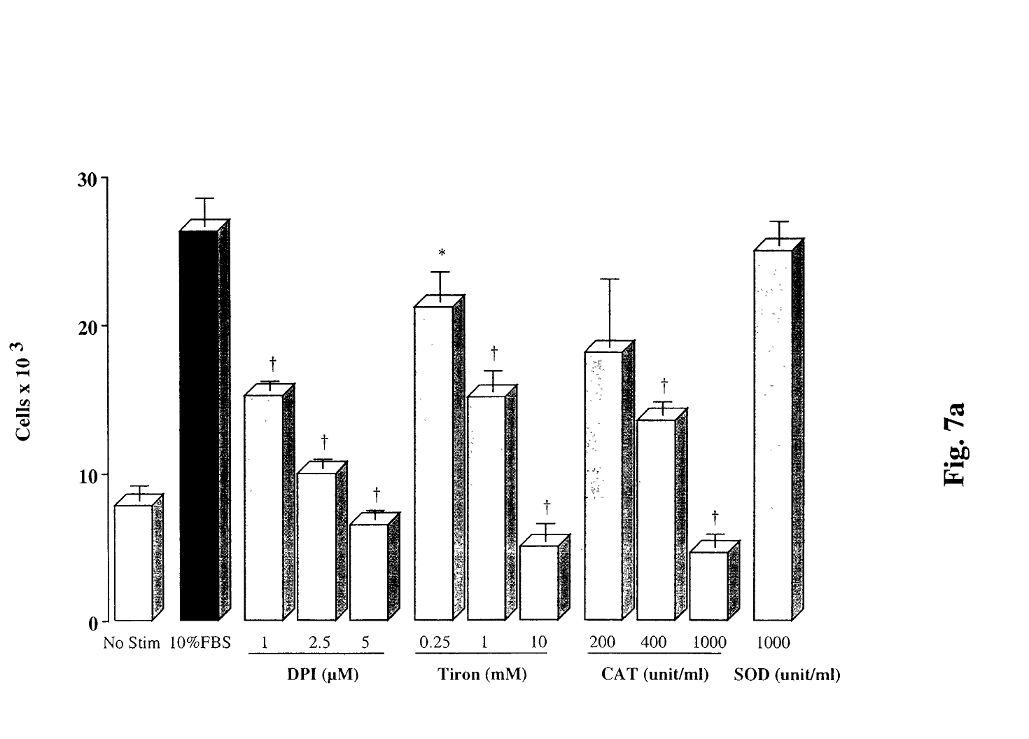

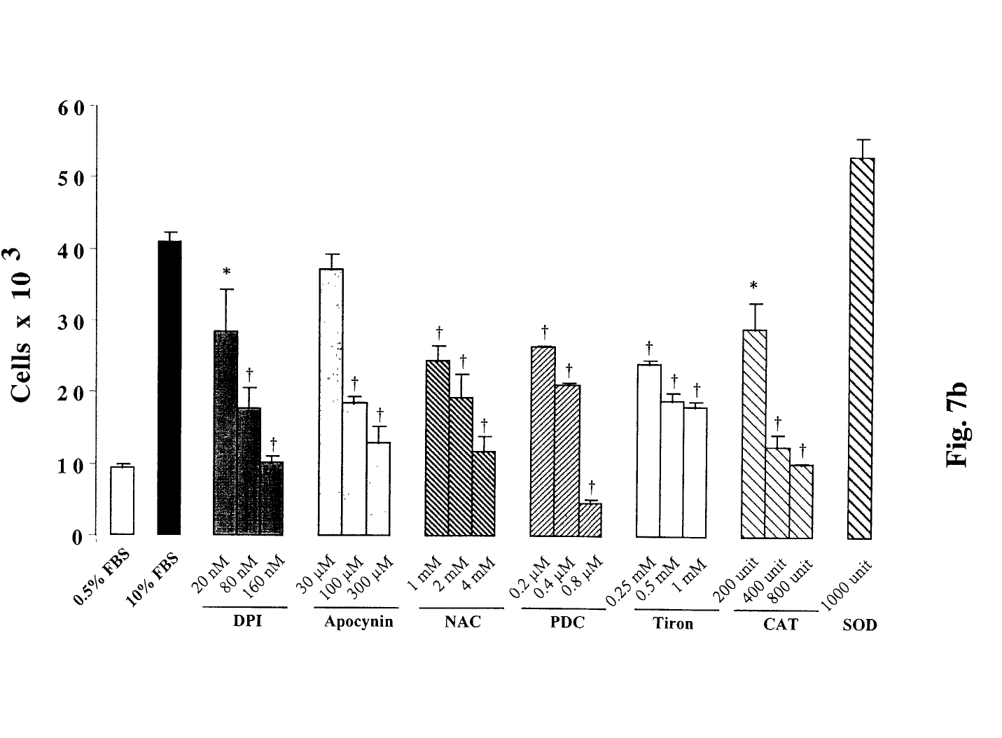

To characterize the mechanism of oxidative stress and its role in cellular proliferation, the present invention focuses on earlier stages of coronary response to injury, with cellular constituents still remaining at sites of their origin. Predominant increases in ROS generation and vascular NAD(P)H oxidase (p47^{phox} and p67^{phox} subunits) are evident in the adventitia (FIGS. 3 and 4). In contrast, coronary media exhibit higher levels of SOD and subsequently lower oxidative stress. It remains to be determined whether the degree of SMC differentiation, which differs among vascular beds, contributes to regional differences in the activation of NAD(P)H oxidase and ROS generation after injury. The inhibition of NAD(P)H oxidase with DPI or the removal of ROS (—O₂⁻ and H₂O₂ with tiron or H₂0₂ with CAT) abrogates serum-induced growth response of isolated vascular cells in vitro (FIGS. 7A and 7B). Not surprisingly, dismutation of —O₂⁻ to H₂O₂ after SOD is ineffective in preventing cell replication, pointing to the essential role of H₂O₂ in the regulation of vascular cell growth. (3)

The results of the present invention imply the involvement of ROS in a rapid proliferation of adventitial fibroblasts after coronary injury in vivo. (22,23,29) The relatively slow and prolonged ROS production in adventitial fibroblasts (FIGS. 2 and 5) is similar to that in non-coronary SMC but quite distinct from the faster and greater response previously seen in phagocytes. (32) Preferential utilization of NADH as a substrate for NAD(P)H oxidase in injured coronary arteries contrasts with the observations by others that aortic adventitial fibroblasts primarily generate —O₂⁻ in response to NAD(P)H. (12,21) Several experimental conditions (e.g., cell origin and type of stimulation), as well as assay methods (e.g., cytochrome c reduction vs. lucigenin assay) may be responsible for these differences. (33)

The increase in oxidative stress stimulates cell growth, but ROS also can cause cellular death. (34) These opposite results are related to the level and the type of ROS (—O₂⁻ versus H₂O₂). (35,36) Much less is known, however, regarding the consequences of oxidative stress in vascular cells, which have a broad range of differentiation. When terminally differentiated cardiomyocytes and interstitial fibroblasts are exposed to H₂O₂, apoptosis is induced in the former, whereas proliferation is induced in the latter. (37) Although endoluminal injury in a porcine model did not significantly enhance intracellular ROS generation in coronary media, extracellular oxidative stress may impact SMC survival. In chronic intimal lesions, inflammatory cells, particularly active in the generation of oxidative stress, have been shown to contribute to SMC apoptosis. (38) These results imply that the loss of differentiated coronary SMC may lead to the decrease of a protective barrier of the intact media, resulting in the expansion of less differentiated fibroblasts and the development of intimal lesions.

The results of the present invention demonstrate that ROS production serves as an attractive target for therapeutic interventions. Nevertheless, several questions remain unresolved, including the choice of antioxidants, since clinical results with vitamin E have been largely negative. (39) In contrast, two independent clinical studies suggest the reduction in coronary restenosis in patients pretreated with the antioxidant probucol prior to angioplasty. (40,41) The recently published HOPE trial also provides evidence for the reduction of cardiovascular mortality after chronic administration of the angiotensin-converting-enzyme inhibitor ramipril. (42) These results are particularly notable since NAD(P)H oxidase activity is regulated by angiotensin II. (10,15,21) Undoubtedly, better understanding of the regulation of NAD(P)H oxidase in different vascular cells may provide further insights into pathogenesis of coronary artery disease and aid the development of therapeutic interventions.

In conclusion, the results of the present invention demonstrate the increase in NAD(P)H oxidase-derived —O₂⁻ production in coronary adventitial fibroblasts after balloon injury. The inhibition of NAD(P)H oxidase and the attenuation of ROS production abrogate proliferative responses of adventitial fibroblasts. The results imply that ROS serve as pivotal signals for growth response of coronary fibroblasts.

### EXAMPLE 2

### Oxidative Stress and Lipid Retention in Vascular Grafts

Methods

### Animal model

A porcine model of a graft interposition in the common carotid artery is used as described. (49) Domestic crossbred pigs (n=22) weighing 35-50 kg are sedated with Telazol (3-5 mg/kg) and atropine (0.01 mg/kg) i.m. Anesthesia is maintained with Propofol, i.v. (15 mg/kg/hr). The animals are ventilated with 100% oxygen. Saphenous veins are harvested without distension and incubated for 30 minutes in saline containing nitroglycerine (0.5 mg/ml). Both carotid arteries are dissected free and heparin (150 U/kg) is administered i.v. A ˜2 cm section of the carotid artery is excised and reversed vein interposition grafting is performed. The excised carotid artery then is grafted into the contralateral carotid artery. Postoperative analgesia is provided with Buprenex (0.015 mg/kg) i.m. The animals are given aspirin 650 mg/day p.o. At the times indicated, animals are euthanized with Euthasol (80 mg/kg) i.v. and vascular tissues harvested.

A separate group of animals (n=14) is placed on the atherogenic diet modified from Weiner et al. (52) at 1 week prior to surgery and continued until graft harvest. Surgical procedure and postoperative care are as described above. These animals demonstrate an increase in serum cholesterol from 79±8 mg/dl at baseline to >250 mg/dl within 3-5 days of the atherogenic diet.

### Measurement of —O₂⁻

Superoxide anion (—O₂⁻) production is measured by SOD-inhibitable conversion of NTB to formazan. (20,21) Normal saphenous veins, arteries, SVG, and AG are harvested at 2 weeks after surgery. After the removal of adventitia and endothelium, tissues are cut into ˜5 mm strips, placed in a 24-well plate, and balanced in pheno-free DMEM at 37° C. in CO₂ for 30 minutes. Freshly made NBT (0.1 mg/ml in pheno-free DMEM) is added for 3 hours with or without addition of SOD (1000 U/ml). The reaction is terminated by 0.5N HCl and rinsing twice with cold PBS. To extract formazan, tissues are pulverized in liquid nitrogen, dissolved in 100% pyridine at 80° C. for 30 minutes, and centrifuged. Supernatants are read at 540 nm and NBT reduction is calculated as follows: NBT reduction=A×V/(T×E×L) (A: absorbance; V: volume of solubilizing solution; T: time of incubation with NBT (minutes); E: extinction coefficient=0.72 mmol.mm; L: length of light travel through the solution (10 mm).) The SOD-inhibitable NBT reduction is calculated as a measure of —O₂⁻ (pmol/mg wet weight/min). In separate experiments, the inhibitors of oxidative enzymes are used, inlcuding diphenyleneiodonium (DPI, 100 μM) rotenone (ROT, 50 μμM), oxypurinol (OXY, 300 μμM) and N^{ω}-nitro-L-arginine methyl ester (L-NAME, 1 mM), to determine the origin of —O₂⁻ in vascular grafts.

### SOD activity

SOD activity in vascular tissue is measured by SOD-dependent inhibition of cytochrome c reduction catalyzed by xanthine/xanthine oxidase. (27,53) After the removal of adventitia and endothelium, vascular media is homogenized in 10 volume of 50 mM potassium phosphate (pH 7.4) containing 0.3 M KBr and a cocktail of protease inhibitors (0.5 mM PMSF, 90 mg/L aprotinin, 10 mg/L pepstatin, 10 mg/L leupeptin). After sonication for 10 seconds, the homogenates are extracted at 4° C. for 30 minutes. The extracts are centrifuged at 20,000 g for 30 minutes. The supernatants are added to a reaction mixture consisting of 0.1 mM EDTA, 0.090 mM xanthine, and 0.018 mM cytochrome c (pH 7.4). SOD activity is assessed by monitoring the inhibition of xanthine oxidase-mediated cytochrome c reduction with absorbance measured at 550 nm over 3 minutes, as described. (9)

### Glycosaminoglycan (GAG) synthesis

Vascular tissues are pulverized in liquid nitrogen, defatted with cold acetone overnight, and dried at 60° C. for 30 minutes. Dry, defatted tissue (DDT) is digested with papain (7 U/ml) in 100 mM sodium acetate, 5 mM cysteine, and 5 mM EDTA at 60° C. for 24 hours. Following precipitation with 0.1% cetylpyridium chloride in 0.1 M sodium citrate (pH 4.8) for 2 hours at 37° C., the pellets are washed with ethanol, air dried, and dissolved in distilled water (100 mg/ml). Sulfated GAG is measured by dye-binding assay (Blyscan, Biocolor LTD, Ireland). Briefly, dye reagent (1,9-dimethylmethylene blue), which is added to the samples, binds to sulfated GAG, thereby forming an insoluble complex. (54) GAG-bound dye is recovered using a dissociation reagent, and the absorbance of the recovered dye is measured in a spectrophotometer at 656 nm. Sulfated GAG (μμg) in vascular tissues is calculated from the calibration curve using the GAG standard. The values are normalized per mg of DDT.

### LDL retention ex vivo

To assess LDL retention in vascular tissues, normal saphenous veins, normal arteries, SVG, and AG are harvested at 14 days after surgery. After the removal of the adventitial and endothelium, vessels are cut into ˜5 mm fragments and placed in 24-well plates. They then are incubated with 125I-labeled LDL (1 mg/ml, 30 cpm/ng) in DMEM (0.5 ml/well) for 24 hours with gentle rocking at 37° C. Tissues are rinsed 5 times (15 min/wash) and blotted dry. Samples are counted in a gamma counter, and values derived from empty wells with ¹²⁵I-labeled LDL are subtracted. LDL retention is expressed per WW (mg), DDT (mg), surface area (mm²), and protein content (ng).

### Immunohistochemistry

The Vectastain Elite ABC system (Vector Laboratories) is used as previously described. (23,28) Tissues are fixed in HistoChoice (Amresco) and processed for paraffin-embedded or frozen sections. They are incubated with primary antibodies for 1 hour, followed by biotinylated secondary horse anti-mouse antibodies (1:2000, Vector Laboratories) for 1 hour. They are visualized with DAB substrate followed by a counterstain with hematoxylin. Monoclonal antibodies against hyaluronate-binding region of human versican (1:200, Developmental Studies Hybridoma Bank), apoB (1:50, Biodesign), and oxidized epitopes (1:50, Biodesign) are used. Negative controls include nonimmune serum instead of primary antibody.

### Statistical analyses

Data are expressed as mean±SE. The statistical significance regarding multigroup comparisons are determined using ANOVA. A value of p<0.05 is considered significant.

### Results

### Redox state in vascular grafts

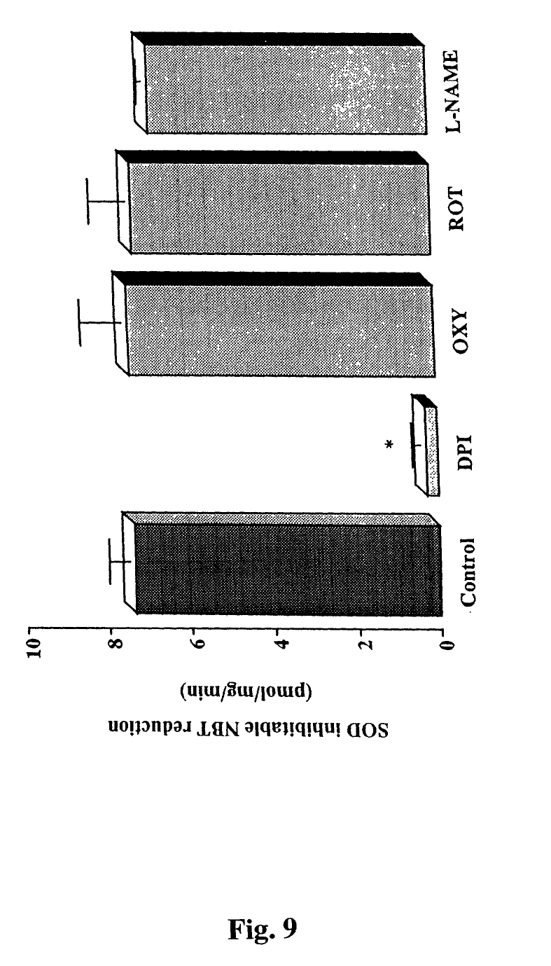

ROS modulate several cellular functions important in vascular remodeling. Accordingly, in the present invention the redox state in venous and arterial grafts is examined by measuring intragraft pro-oxidant and antioxidant properties. As shown in FIG. 8, basal production of —O₂⁻ (SOD-inhibital NBT reduction) is higher in saphenous veins (n=6, p<0.01) than in normal arteries (n=6) prior to grafting. Importantly, vein arterialization further upregulated levels of —O₂⁻ in SVG (n=5, p<0.01) compared to AG (n=7) at 2 weeks after surgery. To determine the source of —O₂⁻ in SVG, several inhibitors of known oxidant enzymes are used. The incubation of SVG with DPI (100 μM, NAD(P)H oxidase inhibitor) almost entirely abolishes —O₂⁻ (reduction by >95%), whereas inhibitors of mitochondrial dehydrogenase (rotenone), xanthine oxidase (oxypurinol), and nitric oxide synthase (L-NAME) show no effects (FIG. 9).

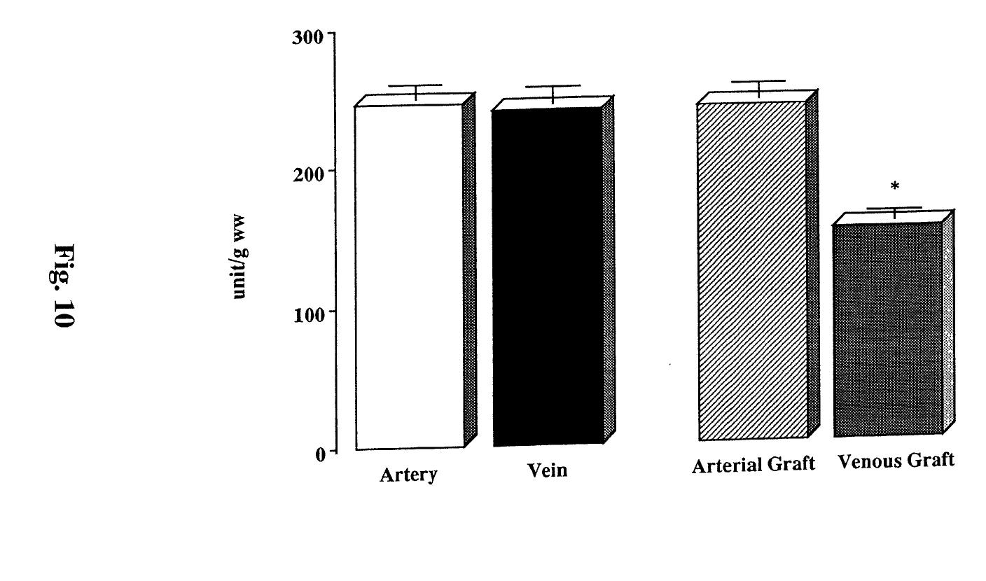

As the graft redox state depends not only on the generation of —O₂⁻ but also on antioxidant properties of the tissue, the affect of arterialization of saphenous veins on SOD activity is examined in the present invention. As illustrated in FIG. 10, normal veins (n=5) and arteries (n=5) demonstrate comparable SOD activity (cytochrome c reduction assay). Nonetheless, SVG (n=8, p<0.001) exhibit a significant loss of SOD activity, whereas AG (n=7) show no changes at 2 weeks after surgery.

### Expression of sulfated GAG and core protein proteoglycans

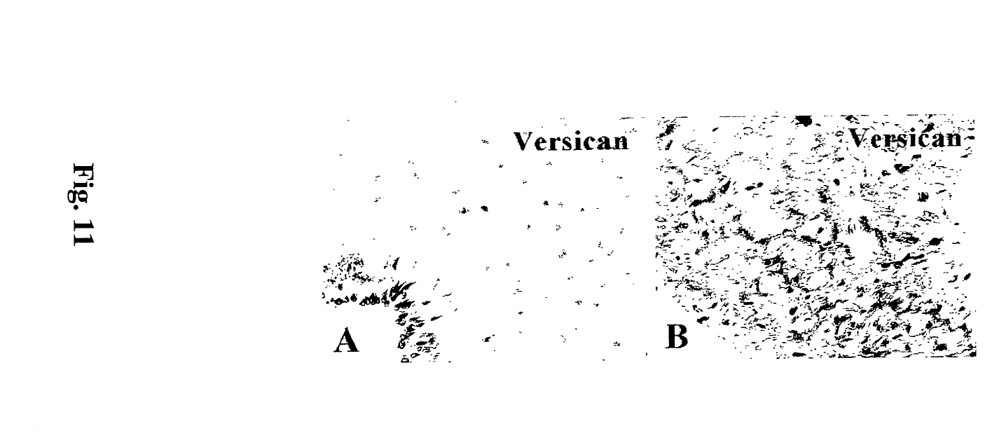

Vascular graft adaptation includes the changes in the extracellular matrix, which may influence the properties of the conduits. To this end the accumulation of sulfated GAG (dye binding assay) in grafts harvested at 2 weeks after surgery is examined. Not surprisingly, normal saphenous veins (n=8, 2.4±0.8 μg/mg DDT) and arteries (n=4, 6.3±0.8 μg/mg DDT) differ in the amount of sulfated GAGs prior to surgery. Importantly, however, vein arterialization (n=8) is accompanied by a significant accumulation of sulfated GAG (3.6±0.8 fold increase, p<0.01 vs. normal vein). In contrast, AG (n=4) show no increase in the amount of GAG (0.68±0.4 fold increase, NS vs. normal artery). As sulfated GAGs constitute side chains of proteoglycans, the above results are further verified by examining the expression and localization of a representative core protein (versican). As shown in FIG. 11, versican immunoreactivity is elevated in the neointima at 2 weeks. In contrast, AG show no apparent changes in versican expression.

### Lipid retention and its modification after grafting

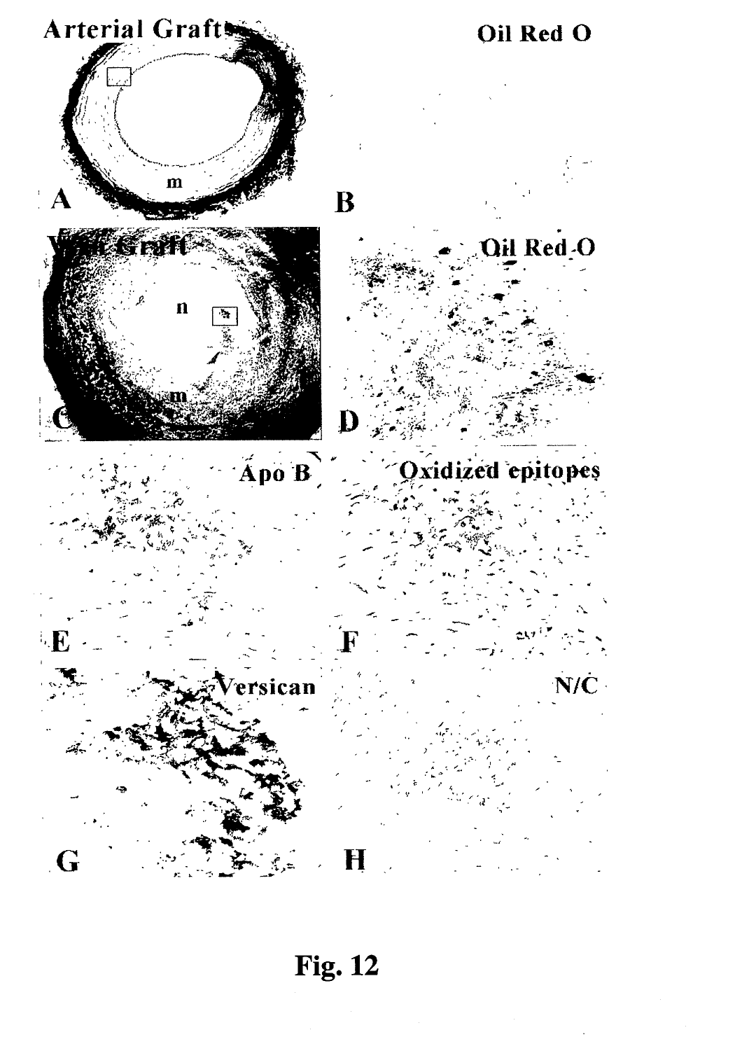

The differences in vessel permeability and its composition (e.g., sulfated GAG content) may increase lipid retention. To address this issue, normal saphenous vein, artery, SVG, and AG are harvested and ¹²⁵I-LDL retention is examined ex vivo. As illustrated in Table 3, intact saphenous veins retain more LDL than arteries, which most likely reflects their dissimilar permeability, although the difference does not reach statistical significance. At 2 weeks after surgery, SVG trap even more radiolabeled LDL over the 24 hour period than do normal saphenous vein, normal artery, or AG (p<0.001) regardless of the method used for data normalization. In contrast, no changes in LDL acumulation are seen in AG. Since the differences in LDL retention in SVG and AG ex vivo do not include hemodynamic factors present in vivo, the intragraft accumulation of lipid in hyperlipemic animals (serum cholesterol 545±49 mg/dl, n−14) is verified. As shown in FIG. 12, AG show no apparent lipid accumulation, whereas SVG contain both extracellular and intracellular deposits of lipid (Red-O-stain). Focal accumulation of apoB and oxidized epitopes is localized in the regions of the neointima containing versican (FIG. 12).

TABLE 3
Retention of ¹²⁵I-LDL in saphenous vein,
normal artery, saphenous vein graft, and arterial graft.
LDL retention (ng)/

| | | mg wet | mg dry | | surface |
|---|---|---|---|---|---|
| Tissue | n | weight | weight | mg protein | area (mm2) |
| Vein | 9 | 118 ± 9 | 0.9 ± 0.06 | 11 ± 2 | 122 ± 8 |
| Artery | 9 | 66 ± 5 | 0.5 ± 0.04 | 6 ± 1 | 56 ± 3 |
| Vein Graft | 20 | 244 ± 13* | 2.9 ± 0.2* | 31 ± 3* | 671 ± 51* |
| Arterial Graft | 9 | 78 ± 7 | 0.6 ± 0.1 | 8 ± 2 | 82 ± 11 |

Normal vessels and vascular grafts 2 weeks after surgery are incubated with ¹²⁵I-LDL (1 mg/ml) as described supra. The values represent mean ± SE, *p < 0.01 vs normal vein, artery, and arterial graft (ANOVA).

### Discussion

Several biologic characteristics distinguish SVG from AG. The arterialization of saphenous veins is marked by a significant shift in the redox state, an accumulation of sulfated proteoglycans, and early lipid retention. The balance between ROS and endogenous antioxidants is an important homeostatic mechanism in vascular tissues. (2) Studies of the arterial system have underscored a preferential generation of —O₂⁻ by adventitial fibroblasts compared with medial SMC. (20,21) The cellular heterogeneity of the venous media, consisting of SMC and fibroblasts, most likely explains the higher levels of —O₂⁻ even under basal conditions, as compared with the arterial media, which is almost exclusively populated with SMC. (49) Future upregulation in —O₂⁻ in arterialized veins could be attributed to several factors. Pulsatile stretch has been implicated in the modulation of oxidative stress. (5) SVG also sustain medical injury during arterialization. (55,56,57) Although focal vascular trauma has been shown to upregulate —O₂⁻ in the arterial system (29,30), studies of vascular grafts have been limited (58). Numerous growth factors released at the site of tissue injury, including thrombin, are known to increase NADH/NAD(P)H oxidase activity. (6) As the results of the present invention demonstrate, an inhibitor of NADH/NAD(P)H oxidase abolishes O₂⁻ production in SVG and the activity of NADH oxidase is augmented in SVG. These findings are consistent with an emerging role of NADH/NAD(P)H oxidase as a primay source of —O₂⁻ in the vasculature. (33)

Two mechanisms that are aimed at removing —O₂⁻ also appear to be impaired in venous grafts. First, venous endothelial cells are less effective than arterial cells in the synthesis of nitric oxide which interacts with —O₂⁻ (46) Second, as shown in the present invention, the overall SOD activity, a major antioxidant enzyme, is attenuated in SVG. It remains to be determined which form of SOD is reduced, although its extracellular form is less abundant in the veins. (51) The observed shift in the redox state could explain the higher cell proliferation seen early after vein arterialization, but generally absent in AG. (49) Furthermore, redox-sensitive transcriptional factors (e.g., NF-KB) may induce the expression of adhesive molecules, such as VCAM-1, which, in turn, promote the influx of blood-borne inflammatory cells into the healing SVG. (59) These mechanisms often lead to the excessive neointimal formation and early occlusive lesions in SVG.

Although vein graft atherosclerosis is clinically manifested several years after surgery, the results of the present invention imply that this process may begin much earlier. Normal saphenous veins retained more LDL ex vivo owing to less developed elastic laminae and likely higher tissue permeability. Although lower venous pressure typically prevents atheroma formation in the venous system in situ, calcified intimal lesions have been occasionally noted in “intact” saphenous veins. (44) As shown in the present invention, LDL retention significantly increases after vein arterialization (2 weeks), but not in AG. This phenomenon was verified in hypercholesterolemic animals, which show lipid retention in the intima of SVG in vivo.

In addition to vessel permeability and hemodynamic factors, vascular lipid retention is affected by extracellular matrix components. Sulfated GAG proteoglycans have been implicated in binding LDL. (60) In particultar, proteoglycans derived from proliferating cells have higher affinity to LDL than those derived from quiescent cells. (61) Previous studies have shown that vascular tissues rich in fibroblasts produce higher amounts of sulfated GAG in conjunction with avid lipid retention, as compared to differentiated SMC. (62) Thus oxidative stress and the synthesis of matrix proteins, which retain LDL, may promote oxidative lipid modifications and create conditions conducive to early onset of SVG atherogenesis.

The results of the present invention demonstrate significant differences in the biology of SVG and AG. Early changes in SVG are characterized by a shift in the redox state due to higher production of —O_{—}⁻ (mediated by NADH oxidase) and lower activity of SOD. Furthermore, SVG increase the synthesis of sulfated GAG proteoglycans which is associated with LDL retention. These findings imply that although SVG atherosclerosis is clinically manifested 3-5 years after surgery, proatherogenic changes may commence early after surgical revascularization.

### Therapeutic uses

The above studies indicate that the administration of NAD(P)H oxidase inhibitors and/or antioxidants can prophylactically and/or therapeutically treat diseases or disorders in a mammal, in particular a human, associated with the abnormal proliferation of cells and extracellular matrix synthesis associated with the activation of NAD(P)H oxidase. In addition to the prevention and treatment of vascular diseases or disorders, such as atherosclerosis, graft disease, and restenosis after revascularization procedures, NAD(P)H oxidase inhibitors and antioxidants are useful for the prevention and treatment of other conditions by decreasing cell proliferation and extracellular matrix synthesis associated therewith. These conditions include, but are not limited to, arthritis, keloid formation, cancer, tissue and organ fibrosis, and complications related to organ transplantation, metabolic syndrome, and radiation therapy.

### Screening

The method comprises administering to a mammal an antioxidant and/or a compound that inhibits NAD(P)H oxidase in an amount sufficient to treat, prophylactically and/or therapeutically, the mammal for diseases, disorders, and conditions associated with the abnormal proliferation of cells and extracellular matrix synthesis associated with the activation of NAD(P)H oxidase. The inhibitor of NAD(P)H oxidase inhibits activation of NAD(P)H oxidase. By activation is meant the change in state of NAD(P)H oxidase from inactive to active. Alternatively, the inhibitor of NAD(P)H oxidase activation inhibits assembly of functional NAD(P)H oxidase, such as by conjugation to essential thiol groups of the membrane-bound and/or cytosolic component(s) of NAD(P)H oxidase. By assembly is meant assembly of the membrane-bound and cytosolic compounds of NAD(P)H oxidase so as to form an active, functional NAD(P)H oxidase.

While it is believed that many of the known NAD(P)H oxidase inhibitors act by interfering with the assembly of the active complex, the term NAD(P)H oxidase inhibitor, as used herein, is not intended to be restricted as to mechanism. Any substance that inhibits the NAD(P)H oxidase-catalyzed generation of reactive oxygen species is encompassed by the term “NAD(P)H oxidase inhibitor”.

Alternatively, the inhibitor of NAD(P)H oxidase is an inhibitor of “oxidative burst”—the intense process by which NAD(P)H oxidase transfers electrons from NAD(P)H to oxygen, resulting in the generation of reactive oxygen species, such as O₂⁻ and H₂O₂. Accordingly, use of the term “NAD(P)H oxidase inhibitor” is intended to encompass all of these compounds, including pharmaceutically acceptable salts thereof, derivatives thereof, dimers thereof, and prodrugs thereof, which can be metabolically converted into an inhibitor of NAD(P)H oxidase or oxidative burst.

Any NAD(P)H oxidase inhibitor can be used in the method of the present invention as long as it is safe and efficacious. Suitable examples of such compounds include those set forth in WO 97/19679 and t'Hart et al., Biotechnology Therapeutics 3 (3 and 4): 119-135 (1992), both of which are specifically incorporated herein in their entireties by reference. While a preferred NAD(P)H oxidase inhibitor in the present invention is apocynin, the method of the invention is not limited to apocynin, and a variety of other chemicals known to inhibit NAD(P)H oxidase in cells may be used, as will be obvious to those skilled in the art. In the case of apocynin, the intact molecule (the ortho-methoxy phenol) is effective as an inhibitor, and in addition, a dimer arising from metabolic oxidation also is highly effective and indeed may be the active species. Thus, compounds having an electron distribution similar to that in the dimer are effective inhibitors of NAD(P)H oxidase and are contemplated within the invention. Examples of other types of NAD(P)H oxidase inhibitors that may be useful include, but are not limited to, isoprenylation inhibitors such as lovastatin and compactin (see U.S. Pat. No. 5,224,916), benzofuranyl- and benzothenylthioalkane carboxylates (see EP application 551,662), and cytochrome b₅₅₈ fragments and their analogs (see PCT application WO 91/17763).

Compounds of the above formula are widely available commercially.

Whether or not a particular compound can inhibit NAD(P)H oxidase can be determined by its effect upon oxygen consumption, NAD(P)H oxidation or radical production, such as production of superoxide, in an assay similar to one of the following assays. Oxygen consumption can be assayed by quantifying changes in oxygen content in a closed system. A decrease in oxygen content represents oxygen utilization by the oxidase system for the production of oxygen free radicals.

Thus, a compound of interest can be combined with a soluble cell fraction (50-150 μμl) and a membrane cell fraction (25-50 μl; equivalent of 2-4×10⁶ cells purified by centrifugation on a discontinuous sucrose gradient) and assay buffer (10 mM Hepes/10 mM potassium phosphate; 0.17 M sucrose; 175 mM NaCl; 0.5 mM EGTA; 1 mM MgCl₂, 10 μμm GTP-γ-S, pH 7.0) at 27° C. Then, 25-100 μμl of sodium dodecyl sulfate (SDS) are added to a final concentration of 100 μμM. The reaction mixture is incubated for 4 minutes and NAD(P)H is added to a final concentration of 200 μμM and the oxygen consumption is recorded at 27° C. using a Clarke electrode. Oxygen consumption indicates assembly and activation of the NAD(P)H oxidase complex. Arachidonic acid, at concentrations determined by the concentrations of the soluble and membrane fractions utilized, can be substituted for SDS. Examples of such assays include those described by t'Hart et al. (Free Radical Biol. Med. 8:241-249, 1990), Bolscher et al. (J. Clin. Invest. 83: 753-763, 1989), Curnette et al. (J. Biol. Chem. 262:5563-5569, 1987), Pilloud et al. (Biochem. Biophys. Res. Comm. 159(2):783-790, 1989) and Doussiere et al. (Biochem. Biophys. Res. Comm. 152(3):993-1001, 1988).

NAD(P)H oxidation can be assayed by monitoring some aspect of the oxidase that is known to undergo a characteristic change upon oxidation. Observation of the characteristic change represents oxidation of NAD(P)H. Typically, this involves spectroscopic evaluation of light absorption at various wavelengths (366 nm for NAD(P)H, 580-530 nm for cytochrome b₅₈₈, and 450-500 nm for flavin oxidoreduction) characteristic of the oxidized or reduced form of a component of the enzyme. Resonance Raman spectroscopies, fluorometric markers of oxidation or absorption decrease at nonoxidized wavelengths as proxy for the rate of oxidation also can be used. Examples of such assays include those described by Koshkin et al. (Biochim. Biophys. Acta 1319:139-146, 1997), Cross et al. (Biochem. J. 194:363-367, 1981; J. Biol. Chem. 270(14):8194-8200, 1995; J. Biol. Chem. 270(29):17075-17077, 1995), Escriou et al. (Eur. J. Biochem. 245(2):505-511, 1997), and Winston et al. (Arch. Biochem. Biophys. 304(2):371-378,1993).

Radical production can be assayed by monitoring the production of superoxide radicals by activated NAD(P)H oxidase in the presence of oxygen and other cofactors. The production of superoxide radicals is proportional to the degree of enzyme activation. Numerous detection and quantification methods are available and include the use of fluorescence, chemiluminescence, electron paramagnetic resonance and spectrophotometric reduction of a marker compound. Examples of such assays include those described by Morel et al. (Biochim. Biophys. Acta 1182:101-109, 1993) and O'Donnell et al. (Biochem. J. 290:41-49, 1993).

Whether or not a particular prodrug can be metabolically converted into an NAD(P)H oxidase inhibitor can be determined in any one of a number of ways. One basic approach is to expose a compound to the various chemical and/or enzymatic milieus to which it will be exposed in the body and to determine whether or not the exposure activates the compound. Then, the ability of the prodrug to inhibit NAD(P)H oxidase can be evaluated in the presence and absence of the chemical/enzymatic milieu. If the prodrug inhibits NAD(P)H oxidase in the presence of the milieu but not in the absence of the milieu, then the prodrug must be converted into an NAD(P)H oxidase inhibitor in the presence of the milieu. The NAD(P)H oxidase inhibiting effect of the prodrug then can be assayed as described above. In this regard, one of ordinary skill in the art will appreciate that prodrugs only can be used in those situations where metabolic conversion to an NAD(P)H oxidase inhibitor is possible.

The NAD(P)H oxidase inhibitor can be bound to a suitable matrix, such as a polymeric matrix, if desired, for use in the present inventive method. Any of a wide range of polymers can be used in the context of the present invention provided that, if the polymer-bound compound is to be used in vivo, the polymer is biologically acceptable.

### Therapeutic/prophylactic methods

One skilled in the art will appreciate that suitable methods of administering an antioxidant and/or a NAD(P)H oxidase inhibitor useful in the method of the present invention are available. Although more than one route can be used to administer a particular antioxidant and/or NAD(P)H oxidase inhibitor, a particular route can provide a more immediate and more effective reaction than another route. Accordingly, the described methods are merely exemplary and are in no way limiting.

The dose administered to an animal, particularly a human, in accordance with the present invention should be sufficient to effect the desired response in the animal over a reasonable time frame. The optimal dose of the NAD(P)H oxidase inhibitor, such as apocynin, or antioxidant to be used in humans will vary depending upon the severity and nature of the condition to be treated, the route of administration, the age, weight, and sex of the patient, as well as on any other medications being taken by the particular patient or the existence of any complicating significant medical conditions of the patient being treated. The dose and perhaps the dose frequency also will vary according to the response of the individual patient. In general, the total daily dose range for apocynin for the conditions described herein is from about 10 mg/kg/day to about 45 mg/kg/day; for the average human, the total dose is about 500 mg to about 3000 mg daily, preferably in divided doses. In managing the patient, the therapy should be initiated at a lower dose, perhaps at about 200 mg to about 500 mg, and increased up to about 1000 mg depending on the patient's global response. It is further recommended that patients over 65 years and those with impaired renal or hepatic function initially receive low doses and that they be titrated based on individual response(s) and blood level(s). It may be necessary to use dosages outside these ranges in some cases, as will be apparent to those skilled in the art. Further, it is noted that the clinician or treating physician will know how and when to interrupt, adjust, or terminate therapy in conjunction with individual patient response. The terms “a therapeutically effective amount” and “an amount sufficient to prevent” a condition are encompassed by the above-described dosage amounts and dose frequency schedule.

Any suitable route of administration may be employed for providing the patient with an effective therapeutic dosage of antioxidant or NAD(P)H oxidase inhibitor. For example, oral, rectal, parenteral (subcutaneous, intramuscular, intravenous), transdermal, aerosol and like forms of administration may be employed.

### Pharmaceutical compositions

Compositions for use in the present inventive method preferably comprise a pharmaceutically acceptable carrier and an amount of an antioxidant and/or NAD(P)H oxidase inhibitor sufficient to treat, either prophylactically or therapeutically, the mammal for diseases, disorders, and conditions associated with the abnormal proliferation of cells and extracellular matrix synthesis associated with the activation of NAD(P)H oxidase. The carrier can be any of those conventionally used and is limited only by chemico-physical considerations, such as solubility and lack of reactivity with the compound, and by the route of administration. It will be appreciated by one of skill in the art that, in addition to the following described pharmaceutical composition, the NAD(P)H oxidase inhibitor and/or antioxidant can be formulated as inclusion complexes, such as cyclodextrin inclusion complexes, or liposomes.

The pharmaceutical compositions of the instant invention may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glycerol monostearate or glycerol distearate may be employed. They may also be coated by the techniques described in the U.S. Pat. Nos. 4,256,108 and 4,265,874 to form osmotic therapeutic tablets for control release.

Pharmaceutical compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Pharmaceutical compositions in the form of aqueous suspensions contain the active materials in admixture with excipients suitable for the manufacture of aqueous suspensions. Such excipients are suspending agents, for example sodium carboxymethyl cellulose, methylcellulose, hydroxypropylmethylcellulose, sodium alginate, polyvinylpyrrolidone, gum tragacanth and gum acacia; dispersing or wetting agents, which may be a naturally-occurring phosphatide, for example lecithin, or condensation products of an alkylene oxide with fatty acids, for example polyoxyethylene stearate, or condensation products of ethyylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Pharmaceutical compositions in the form of oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Pharmaceutical compositions that are dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions for use in the above methods of the invention also may be in the form of oil-in-water emulsions. The oily phase may be a vegetable oil, for example olive oil or arachia oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids an hexicol-anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs are also suitable pharmaceutical formulations for use is the instant methods. Such syrups and elixirs may be formulated with sweetening agents, for example glycerol, propylene glycol, sorbitol or sucrose. Such compositions may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents that have been mentioned above. The sterile injectable preparation may also be a sterile injectable solution or suspension in a non-toxic parenterally-acceptable diluent or solvent, for example as a solution in 1,3-buzane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The pharmaceutical compositions of the present invention may be administered in the form of suppositories for rectal administration of the drug. These compositions can be prepared by mixing the drug with a suitable non-irritating excipient that is solid at ordinary temperatures but liquid at the rectal temperature and will therefore melt in the rectum to release the drug. Such materials are cocoa butter and polyethylene glycols.

For topical use, pharmaceutical compositions comprising creams, ointments, jellies, solutions or suspension, etc., containing the antioxidants and/or NAD(P)H oxidase inhibitors of the present invention are employed in the instant methods. (For purposes of this application, topical application shall include mouth washes and gargles.) Topically-transdermal patches also are included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservations, absorption promoters to enhance bioavailability, fluorocarbons, and/or other solubilizing or dispersing agents known in the art.

The present inventive method also can involve the co-administration of other pharmaceutically active compounds. By “co-administration” is meant administration before, concurrently with, or after administration of an antioxidant and/or NAD(P)H oxidase inhibitor as described above.

We claim:

1. A method for the prophylactic and/or therapeutic treatment of a disease or disorder associated with abnormal proliferation of cells and extracellular matrix synthesis associated with activation of NAD(P)H oxidase comprising:
(a) administering a therapeutically effective amount of an NAD(P)H oxidase inhibitor(s);
(b) inhibiting the synthesis or translocation of NAD(P)H oxidase subunits;
(c) blocking the generation of intracellular ROS in said cells;
(d) inhibiting the proliferation of said cells and extracellular matrix synthesis; and
(e) prophylactically and/or therapeutically treating said disease or disorder.

2. The method of claim 1, wherein said disease or disorder is a vascular disease or disorder.

3. The method of claim 2, wherein said vascular disease or disorder is at least one of the group comprising atherorsclerosis, graft disease, and restenosis after revascularization procedures.

4. The method of claim 1, wherein said cells are at least one of smooth muscle cells and fibroblasts.

5. The method of claim 1, wherein said NAD(P)H oxidase inhibitor is DPI.

6. The method of claim 1, wherein said NAD(P)H oxidase inhibitor is apocynin.

7. A method for the prophylactic and/or therapeutic treatment of a disease or disorder associated with ROS mediated abnormal proliferation of cells and extracellular matrix synthesis comprising:
(a) administering a therapeutically effective amount of an antioxidant(s);
(b) blocking the generation of intracellular ROS in said cells;
(c) inhibiting the proliferation of said cells and extracellular matrix synthesis; and
(d) prophylactically and/or therapeutically treating said disease or disorder.

8. The method of claim 7, wherein said disease or disorder is a vascular disease or disorder.

9. The method of claim 8, wherein said vascular disease or disorder is at least one of the group comprising atherorsclerosis, graft disease, and restenosis after revascularization procedures.

10. The method of claim 7, wherein said cells are at least one of smooth muscle cells and fibroblasts.

11. The method of claim 7, wherein said antioxidant is at least one of the group comprising N-acetylcysteine, pyrrolidinedinedithiocarbamate, tiron, catalase, and glutathione.

12. A method for the prophylactic and/or therapeutic treatment of a disease or disorder associated with abnormal proliferation of SMC and/or fibroblasts and extracellular matrix synthesis associated with activation of NAD(P)H oxidase comprising:
(a) administering a therapeutically effective amount of an NAD(P)H oxidase inhibitor(s);
(b) inhibiting the synthesis or translocation of NAD(P)H oxidase subunits;
(c) blocking the generation of intracellular ROS in said SMC and/or fibroblasts;
(d) inhibiting the proliferation of said SMC and/or fibroblasts and extracellular matrix synthesis; and
(e) prophylactically and/or therapeutically treating said disease or disorder.

13. The method of claim 12, wherein said disease or disorder is a vascular disease or disorder.

14. The method of claim 13, wherein said vascular disease or disorder is at least one of the group comprising atherorsclerosis, graft disease, and restenosis after revascularization procedures.

15. A method for the prophylactic and/or therapeutic treatment of a vascular disease or disorder associated with abnormal proliferation of SMC and/or fibroblasts and extracellular matrix synthesis associated with activation of NAD(P)H oxidase comprising:
(a) administering a therapeutically effective amount of an NAD(P)H oxidase inhibitor(s);
(b) inhibiting the synthesis or translocation of NAD(P)H oxidase subunits;
(c) blocking the generation of intracellular ROS in said SMC and fibroblasts;
(d) inhibiting the proliferation of said SMC and fibroblasts and extracellular matrix synthesis; and
(e) prophylactically and/or therapeutically treating said disease or disorder.

16. A method for the prophylactic and/or therapeutic treatment of a disease or disorder associated with ROS mediated abnormal proliferation of SMC and/or fibroblasts and extracellular matrix synthesis comprising:
(a) administering a therapeutically effective amount of an antioxidant(s);
(b) blocking the activity of intracellular ROS in said cells;
(c) inhibiting the proliferation of said SMC and/or fibroblasts and extracellular matrix synthesis; and
(d) prophylactically and/or therapeutically treating said disease or disorder.

17. The method of claim 16, wherein said disease or disorder is a vascular disease or disorder.

18. The method of claim 17, wherein said vascular disease or disorder is at least one of the group comprising atherorsclerosis, graft disease, and restenosis after revascularization procedures.

19. A method for the prophylactic and/or therapeutic treatment of a vascular disease or disorder associated with ROS mediated abnormal proliferation of SMC and/or fibroblasts and extracellular matrix synthesis comprising:
(a) administering a therapeutically effective amount of an antioxidant(s);
(b) blocking the generation of intracellular ROS in said SMC and/or fibroblasts;
(c) inhibiting the proliferation of said SMC and/or fibroblasts and extracellular matrix synthesis; and
(d) prophylactically and/or therapeutically treating said disease or disorder.

20. A pharmaceutical composition comprising a pharmaceutically acceptable carrier and a therapeutically effective amount of at least one NAD(P)H oxidase inhibitor.

21. A pharmaceutical composition comprising a pharmaceutically acceptable carrier and a therapeutically effective amount of at least one antioxidant.
